(19) United States
(12) Patent Application Publication
(10) Pub. No.: US 2021/0282686 A1
(43) Pub. Date: Sep. 16, 2021

(54) SYSTEM AND METHOD FOR PROVIDING PERSONALIZED RECOMMENDATIONS FOR DAILY STRESS MANAGEMENT BASED ON USER RESPONSE HISTORY AND DATA FROM WEARABLES

(71) Applicant: KONINKLIJKE PHILIPS N.V., EINDHOVEN (NL)

(72) Inventors: Akhil CHATURVEDI, Pittsburgh, PA (US); Gary Nelson GARCIA MOLINA, Madison, WI (US); Boomika KALYAN, Pittsburgh, PA (US); Terese NAVARRA, Pittsburgh, PA (US); Mantek CHADHA, Cambridge, MA (US); Gaurav TRIVEDI, Pittsburgh, PA (US)

(21) Appl. No.: 17/201,038

(22) Filed: Mar. 15, 2021

Related U.S. Application Data
(60) Provisional application No. 62/990,110, filed on Mar. 16, 2020.
(60) Provisional application No. 63/046,372, filed on Jun. 30, 2020.

(51) Int. Cl. A61B 5/16; G16H 50/30; G16H 50/70; G16H 20/70; G16H 10/65; G06N 3/08

(52) U.S. Cl. CPC A61B 5/165; G16H 50/30; G06N 3/08; G16H 20/70; G16H 10/65; G16H 50/70

(57) ABSTRACT

A method of detecting and treating stress in a patient includes receiving in a controller patient information obtained passively from one or more electronic devices associated with the patient; analyzing the patient information with the controller; determining in the controller, from the analyzing of the patient information, a stress-reducing treatment for the patient; and providing the stress-reducing treatment to the patient.

### CROSS-REFERENCE TO PRIOR APPLICATIONS

This application claims the benefit of U.S. Provisional Application No. 62/990110, filed on 16 Mar. 2020 and U.S. Provisional Application No. 63/046372, filed on 30 Jun. 2020. These applications are hereby incorporated by reference herein.

### BACKGROUND OF THE INVENTION

### 1. Field of the Invention

The present invention relates to reducing stress in an individual and, more particularly, to methods of detecting and treating stress in an individual. The present invention also relates to systems for carrying out methods for detecting and treating stress in an individual.

### 2. Description of the Related Art

Increased stress and deteriorating mental health have become major concerns worldwide. Excessive, unmanaged chronic stress severely and negatively affects overall health. Modern busy lifestyles have led to inappropriate regulation of stress response, which is linked to a wide range of pathologies. Real time stress detection techniques using physiological signals like heart rate and skin conductivity are widely discussed in literature (Zhai and Barreto, 2006; De et al., 2011; Smets, De Raedt and Van Hoof, 2019). Stress prediction algorithms based on feature extraction have been studied (Sriramprakash, Prasanna and Murthy, 2017). Standalone solutions like paced breathing, mindfulness techniques, bilateral stimulation, etc. have made their way into the realm of relaxation solutions, however, response to a stimulus like stress differs by every individual and needs to be addressed accordingly. Response to a stimulus is often associated with the experience that the user has had, in that situation, i.e., a smell or a sound, feeling of fear/anxiety or happiness, snippets from visual memory, etc. It is important to identify the situations that collectively result in an uncontrolled stress response, and the situations that result in a relaxed response, to be able to manage the stress response better.

### SUMMARY OF THE INVENTION

Embodiments of the present invention improve over conventional arrangements and methods by providing personalized recommendations/solutions for reducing stress in a particular individual. As one aspect of the present invention a method of detecting and treating stress in a patient is provided. The method comprises: receiving in a controller patient information obtained passively from one or more electronic devices associated with the patient; analyzing the patient information with the controller; determining in the controller from the analyzing of the patient information a stress-reducing treatment for the patient; and providing the stress-reducing treatment to the patient.

The patient information may comprise biometric information of the user.

The biometric information of the user may comprise one or more of: voice pitch, heart rate variability, breathing rate, body movement, skin conductivity, and/or galvanic skin response.

The controller may be structured and configured to implement a predictive AI system, and the analyzing and determining may be performed by the predictive AI system.

The predictive AI system may be an artificial neural network trained using one or more of: previous patient information about the patient and/or patient information about a number of other patients.

The one or more electronic devices associated with the patient may comprise a wearable smart device.

The one or more electronic devices associated with the patient may comprise a smartphone.

The patient information may comprise environmental information regarding one or more details of an environment in which the user is disposed.

The environmental information may comprise one or more of: weather, location, and/or ambient illumination.

The patient information may comprise social information of the patient, and the social information may comprise one or more of: local traffic condition, meeting schedule, workload, social media state, sleep/wake timing, phone useage, and/or typing speed.

The method may further comprise receiving additional patient information actively from the patient.

The additional patient information may be received from the one or more electronic devices associated with the patient.

The additional patient information may be obtained from another device other than the one or more electronic devices associated with the patient.

The method may further comprise determining a stress level of the patient is one of above or below a predetermined range, and responsive thereto, prompting the patient to provide contextual information for associating with the stress level above or below the predetermined range.

The stress-reducing treatment may comprise one or more of: paced breathing, taking a break from a current activity, a short walk, meditation, listening to music, and or aromatherapy.

As another aspect of the present invention, a system for detecting and treating stress in a patient is provided. The system comprises: a number of electronic devices, each structured to passively capture patient information from the patient; a controller in communication with the number of electronic devices and structured to carry out an analysis of the patient information and determine a stress-reducing treatment for the patient based on the analysis of the patient information; and an output device structured to convey the stress-reducing treatment to the patient.

As yet a further aspect of the present invention, a predictive artificial intelligence system is provided. The predictive artificial intelligence system being trained to: receive patient information obtained passively from one or more electronic devices associated with a patient; analyze the patient information; determine a stress-reducing treatment for the patient based on the analysis of the patient information; and provide the stress-reducing treatment to the patient.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a method for detecting and treating stress in a patient in accordance with one example embodiment of the present invention; and

FIG. 2 is a schematic representation of a system in accordance with one example embodiment of the present invention that may be employed in carrying out the method of FIG. 1.

### DETAILED DESCRIPTION OF EXEMPLARY EMBODIMENTS

As used herein, the singular form of “a”, “an”, and “the” include plural references unless the context clearly dictates otherwise.

As used herein, the term “number” shall mean one or an integer greater than one (i.e., a plurality).

As used herein, the terms “user”, “patient”, and “individual” are used interchangeably to refer to a person who is being generally monitored for stress and being provided with stress-reducing treatments in accordance with embodiments of the present invention.

As used herein, the term “controller” shall mean a number of programmable analog and/or digital devices (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

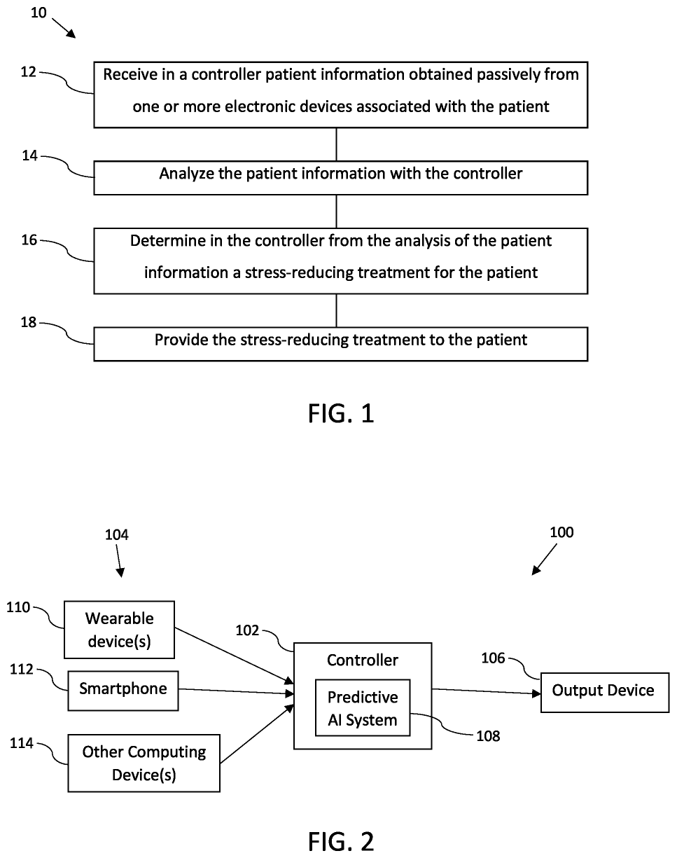

As previously discussed, excessive, unmanaged chronic stress severely and negatively affects overall health. Depending on the type of stressful situations, different relaxation strategies may be used to promote relaxation. However, there exists no single stress management strategy capable of coping with any stressful situation for a given individual. Instead, different stress management strategies have different effects in addressing different stress inducers in different individuals. Embodiments of the present invention provide stress management systems and methods based on a predictive model that utilizes several parameters like biometric data (users' history of reactions to stressful or joyous situations), environmental data (location, weather, traffic, etc.) and self-annotations (stress diary). The model provides personalized recommendations to be able to manage stress most effectively, as observed from prior “relaxed” responses. An example method 10 for detecting and treating stress in a patient in accordance with one example embodiment of the present invention is shown in FIG. 1 and a schematic representation of a system 100 in accordance with one example embodiment of the present invention that may be employed in carrying out method 10 is shown in FIG. 2.

System 100 includes a controller 102 structured to receive input from one or more of a number of input devices 104 and provide output to one or more of a number of output devices 106. Alternatively, one or more of the number of input devices 104 may function as both and input and output device and thus both provide input to, and receive output from, controller 102. As generally shown in step 12 of method 10, such input provided to controller 102 includes information about a patient/user of system 100 which is hereinafter referred to as “patient information”. As discussed further below, such patient information may be obtained passively or actively by one or more of input devices 104 and may contain one or more of: information about biometrics of the patient (hereinafter “biometric information”), information about the environment in which the patient is disposed (hereinafter “environmental information), and/or information about the social activities in which the patient is involved (hereinafter “social information”).

Controller 102 may be provided locally as a computing device or (portion thereof) or remotely as a cloud based arrangement. A memory portion of controller 102 has stored therein a number of routines that are executable by a processor portion of controller 102. One or more of the aforementioned routines implement (by way of computer/processor executable instructions) a software application that is configured (by way of one or more algorithms) to, among other things, receive input from one or more of the number of input devices 104 and analyze such input in order to determine the stress level of the user and when appropriate, provide a stress-reducing treatment to be carried out by the user. In the example embodiment of FIG. 2, controller 102 is provided with a predictive AI system 108, such as a trained neural network or other supervised learning systems, for this purpose. In such an embodiment, training of predictive AI system 108 can be done by collecting patient information of patients and manually categorizing such information into being indicators of different levels of stress or causes thereof. Predictive AI system 104 can be further trained by collecting further patient information after various stress-reducing treatments have been carried out along with the information regarding the particular stress-reducing treatment(s) carried out. As generally shown in steps 14, 16 and 18 of method 10, once trained, predictive AI system 108 will recognize and/or predict an elevated stress level of a patient/user from patient information received as input from one or more of input devices 104 and output a suggested stress reducing treatment via output device 106 to be carried out by the patient. Over time, such arrangement “learns” what works best for a particular patient and thus will provide optimum stress-reducing treatments for the particular patient.

Referring again to FIG. 2, the number of input devices 104 of system 100 may include a number of wearable devices 110 for detecting biometric information of the patient/user and transmitting such patient information to controller 102 (and thus to predictive AI system 108 thereof). Each wearable device 110 may be of any suitable arrangement (e.g., without limitation, a smartwatch, a smart ring, a dedicated monitoring device, etc.) that is structured to detect and passively or actively transmit one or more biometrics of the user to controller 102. Such biometric information of the user may include one or more of: heart rate (or variability thereof), breathing rate, body movement, skin conductivity, galvanic skin response, and/or voice pitch. In addition to, or in place of, wearable device 110, biometric information of the user may be detected and transmitted to controller 102 via a smartphone 112 provided as one of the number of input devices 104 of system 100. In addition to, or in place of one or both of wearable device(s) 110 and/or smartphone 112, system 100 may include one or more other computing devices 114 (e.g., without limitation a tablet computer, laptop computer, desktop computer, etc.) for providing patient information or other input to, or receiving output from, controller 102. For example, environmental information for the patient, e.g., without limitation, weather, location, ambient noise, and/or ambient illumination may be provided to controller 102 via sensors (not numbered) provided on or in one or more of input devices 104. Such environmental information may also be provided in-part from such sensors used in conjunction with other resources (e.g., without limitation, weather conditions provided/determined using GPS sensor in conjunction with location information provided by the National Weather Service). As another example, social information for the patient, e.g., without limitation, local traffic condition, meeting schedule, workload, social media state, sleep/wake timing, phone useage, and/or typing speed may be provided to controller 102 in whole or in-part by one of input devices 104.

From the foregoing it is thus to be appreciated that system 100 generally monitors/records the level of stress of a user using sensors which detect biometrics of the user. As the user's real time stress information is monitored/recorded, system 100 also records and classifies stimuli and input from the user's environment (i.e., environmental information and/or social information) to generally develop an environmental scene classification. In addition to patient information passively obtained, on detection of abnormally high stress levels or a particularly well relaxed state, the user (provided they are awake and willing) may be prompted to provide contextual information (e.g., via one of input devices 104) to enable system 100 (and particularly Predictive AI System 108) to learn about the most impactful stimuli or situation influencing the user's stress profile. By using models to detect both the real time stress state of the user as well as the environmental conditions, combinations of detected variables in a user's environment are correlated with a user's stress state over time and multiple episodes of stress/relaxation activity. The data for each user is stored as a personalized dictionary of Environmental variables and stress intensities so that we have a personalized metric of environmental situations for each user which can be related to the stress profile of each user. Given stress profiles for each individual user, under the assumption of having an exhaustive number of relaxation techniques, machine learning models are used to create relaxation techniques at different time points, leading to more effective intervention strategy to combat stress than conventional solutions.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of detecting and treating stress in a patient, the method comprising:
receiving in a controller patient information obtained passively from one or more electronic devices associated with the patient;
analyzing the patient information with the controller;
determining in the controller from the analyzing of the patient information a stress-reducing treatment for the patient; and
providing the stress-reducing treatment to the patient.

2. The method of claim 1, wherein the patient information comprises biometric information of the user.

3. The method of claim 2, wherein the biometric information comprises one or more of: voice pitch, heart rate variability, breathing rate, body movement, skin conductivity, and/or galvanic skin response.

4. The method of claim 1, wherein the controller is structured and configured to implement a predictive AI system, and wherein the analyzing and determining is performed by the predictive AI system.

5. The method of claim 4, wherein the predictive AI system is an artificial neural network trained using one or more of: previous patient information about the patient and/or patient information about a number of other patients.

6. The method of claim 1, wherein the one or more electronic devices associated with the patient comprise a wearable smart device.

7. The method of claim 1, wherein the one or more electronic devices associated with the patient comprises a smartphone.

8. The method of claim 1, wherein the patient information comprises environmental information regarding one or more details of an environment in which the user is disposed.

9. The method of claim 8, wherein the environmental information comprises one or more of: weather, location, ambient illumination, and/or ambient noise.

10. The method of claim 1, wherein the patient information comprises social information of the patient, and wherein the social information comprises one or more of: local traffic condition, meeting schedule, workload, social media state, sleep/wake timing, phone useage, and/or typing speed.

11. The method of claim 1, further comprising receiving additional patient information actively from the patient.

12. The method of claim 1, further comprising determining a stress level of the patient is one of above or below a predetermined range, and responsive thereto, prompting the patient to provide contextual information for associating with the stress level above or below the predetermined range.

13. The method of claim 1, wherein the stress-reducing treatment comprises one or more of: paced breathing, taking a break from a current activity, a short walk, meditation, listening to music, and or aromatherapy.

14. A system for detecting and treating stress in a patient, the system comprising:
a number of electronic devices, each structured to passively capture patient information from the patient;
a controller in communication with the number of electronic devices and structured to carry out an analysis of the patient information and determine a stress-reducing treatment for the patient based on the analysis of the patient information; and
an output device structured to convey the stress-reducing treatment to the patient.

15. A predictive artificial intelligence system trained to:
receive patient information obtained passively from one or more electronic devices associated with a patient;
analyze the patient information;
determine a stress-reducing treatment for the patient based on the analysis of the patient information; and
provide the stress-reducing treatment to the patient.
